(19) United States
(12) Patent Application Publication
(10) Pub. No.: US 2007/0065954 A1
(43) Pub. Date: Mar. 22, 2007

(54) Surface plasmon resonance biosensor system for detection of antigens and method for determining the presence of antigens

(76) Inventors: Minoru Taya, Mercer Island, WA (US); Fengyu Su, Kenmore, WA (US); Chunye Xu, Seattle, WA (US); Kazuko Handa, Bellevue, WA (US); Senitiroh Hakomori, Mercer Island, WA (US); Kimie Murayama, (US)

Correspondence Address: SUGHRUE MION, PLLC, 2100 PENNSYLVANIA AVENUE, N.W., SUITE 800, WASHINGTON, DC, 20037, US

(21) Appl. No.: 11/520,663

(22) Filed: Sep. 14, 2006

Related U.S. Application Data
(60) Provisional application No. 60/716,929, filed on Sep. 15, 2005.

(51) Int. Cl. G01N 33/551

(52) U.S. Cl. 436/524

(57) ABSTRACT

The present invention provides an SPR system and corresponding methods of use, for determining the presence or concentration of tumor-associated antigens in cancer patient samples. The SPR system may have multiple channels, with each channel having operably affixed thereto an antibody specific for a tumor-associated antigen, so as to allow detection of multiple tumor-associated antigens simultaneously. When a biological sample from a patient is applied to the SPR system, the presence of two or more tumor-associated antigens can be determined by measuring an SPR signal shift from each channel. The SPR system may detect the presence or concentration of a tumor-associated carbohydrate antigen, where the sensor surface contains affixed thereto an antibody specific for the glycosyl epitope, as well as an antibody specific for the polypeptide to which the carbohydrate antigen is naturally associated in cancer patients.

This application is a non-provisional application claiming the benefit of Provisional Application No. 60/716,929, filed Sep. 15, 2005, which is hereby incorporated by reference in its entirety.

### FIELD OF THE INVENTION

The present invention relates to a surface plasmon resonance (SPR) system and method for determining the presence or concentration of antigens in patient samples, to thereby improve the diagnosis and prognosis of disease, and particularly cancer.

### BACKGROUND OF THE INVENTION

Detection of cancer at its early stage is essential for successful treatment. Many tumor-associated antigens are elevated in patient sera, and are thus useful as diagnostic targets. However, while attempts have been made to apply these tumor-associated antigens for diagnosis and prognosis of cancer (Hakomori, S. Adv. Cancer Res. 52, 257-331, 1989; Adv. Exp. Med. Biol. 491, 369-402, 2001), there are several hurdles to the creation of an effective and broadly applicable test.

First, detecting cancer at an early stage requires sensitive analytic means. For example, carcinoembryonic antigen (CEA) is a tumor-associated protein antigen, which has been used as a tumor marker for diagnostic and therapeutic purposes in various neoplasias, such as gastrointestinal, breast and lung cancer (Aquino et al., Pharmacol. Res. 49, 383-396, 2004). CEA is typically present in an adult non-smoker at <2.5 ng/ml, and <5.0 ng/ml for smokers. Thus, a suitable analytical means for detecting CEA must provide sensitive detection at this concentration range.

Second, a single tumor-associated antigen is insufficient for all diagnoses. A single tumor expresses multiple tumor-associated antigens (“mosaicism”), and the antigen expression pattern changes during cancer development (Nakasaki, H., Hakomori, S. et al., Cancer Res. 49, 3662-9, 1989). Some tumor-associated antigens may even be more effective in certain populations. For example, the black population has a high incidence of the Le(a-b-) genotype, and therefore, tumors in this population have limited expression of the sialyl-Le^{a} (SLe^{a}) antigen.

It is therefore also highly desirable to determine the presence or concentration of multiple tumor-associated antigens in serum from a single patient. Tumor-associated antigens in serum have been conventionally determined by immunoassay, and in particular fluorescent, enzymatic and radioimmunoassay, in which the amount of antigen-antibody complex is determined by labeled secondary antibodies. Using the conventional techniques, determination of multiple antigens within a single sample is technically difficult, and the cost of such determination rises in proportion to the number of antigens tested.

Similarly, it is costly to run a separate test for each patient sample, and thus methods and systems for testing multiple patient samples more efficiently are needed.

Alternative analytical means for detecting the presence of analytes include Surface Plasmon Resonance (SPR) biosensors (Liedberg B. et al., Biosensors & Bioelectronics 10, i-ix, 1995; Homola et al., Sensors and Actuators B 54, 3-15, 1999; Karlsoon et al., Methods 9, 99-110, 1994). SPR is an optical phenomenon occurring at the interface between a metal and a dielectric medium, and is sensitive to changes in thickness and refractive index of a thin analyte layer on the metal surface. Thus, an SPR biosensor can determine a refractive index change, or SPR signal shift, due to an interaction between a ligand and an analyte at the sensor surface.

An SPR signal shift occurs due to the thickness of molecules bound to the matrix, and requires a mass of analyte to bind to the sensor surface. Thus, sensitive detection via SPR will depend on the thickness of the analyte layer.

In terms of cancer detection with SPR, antigens such as HER-2 and NY-ESO-1 have been immobilized to the sensor surface for detection of tumor-associated antibodies in patient samples (Russel et al, 225^{th} ACS National Meeting, New Orleans, La., Mar. 23-27, 2003; Campagnolo et al., J Biochem. Biophys. Methods 61, 283-298, 2004).

However, the ability of SPR to detect smaller molecular weight analytes, those of unknown size, and/or those present at relatively low concentrations in patient samples has not been determined.

In this respect, various tumor-associated antigens were originally defined by monoclonal antibodies, with many of the epitopes later being identified as glycosphingolipids. These antigens typically have molecular weights in the range of 2,000 to 4,500 Da, but are presumably associated with lipoproteins or other protein complexes in serum, such that there actual molecular weights are unknown and presumably much higher. It is therefore unknown whether the glycosylsphingolipid epitope will be suitable for SPR analysis of tumor-associated carbohydrate antigens, given questions concerning the mass and concentration of the antigen in serum. Table 1 summarizes some tumor-associated carbohydrate antigens, and illustrates their structure.

An anaytical system suitable for detecting the presence or concentration of tumor-associated antigens, such as antigens present at low concentrations in patient samples or carbohydrate antigens, is needed. Further, a diagnostic system to allow for convenient and cost effective analysis of multiple antigens or multiple samples simultaneously, is of great interest for early cancer detection and improving the survival of patients.

### SUMMARY OF THE INVENTION

It is an object of the present invention to provide a system and method for determining the presence or concentration of a tumor-associated antigen in a biological sample.

Thus, one aspect of the invention provides an SPR system. Preferably, the SPR system is capable of detecting multiple antigens simultaneously, and therefore has multiple channels, with each channel having operably affixed thereto an antibody specific for a tumor-associated antigen. When a biological sample from a patient is applied to the SPR system, the presence of two or more tumor-associated antigens can be determined by measuring an SPR signal shift from each channel.

In a preferred aspect of the invention, the SPR system detects the presence of a tumor-associated carbohydrate antigen. In a particularly preferred embodiment, the sensor surface contains affixed thereto an antibody specific for the glycosyl epitope, as well as an antibody specific for the polypeptide to which the carbohydrate antigen is naturally associated in cancer patients. The antibodies are preferably Fab fragments.

Another aspect of the invention provides a method for determining the presence of a tumor-associated antigen by employing the SPR system.

The invention will now be described in greater detail below.

### BRIEF DESCRIPTION OF DRAWINGS

FIG. 1 illustrates that multiple antigens are expressed in different loci of a single primary human tumor. Panel I, primary colonic cancer; Panel II, gastric cancer; Panel III, well-differentiated gastric cancer.

FIG. 2 shows a Western blot analysis with anti-sialyl-Le^{x} (SNH3) of sera from normal subjects, and of sera from lung cancer patients. The left panel shows staining for total protein (Coomassie Brilliant Blue). The right panel shows the Western analysis with anti-sialyl-Le^{x} (SNH3).

FIG. 3 illustrates exemplary SPR biosensor systems. FIG. 3A illustrates a compact SPR biosensor including a sample cassette with disposable sensor chip. FIG. 3B illustrates an SPR system based on Kretschmann configuration. FIG. 3C shows surface functionalization of a sensor chip (a), and the immobilization of CEA antibodies to the sensing surface (b).

FIGS. 4A-4F illustrate the concept and assembly of a self-referencing SPR system.

FIGS. 5A and B illustrate the absorption of mouse IgG and TKH2 antibody onto a gold surface in the construction of a self-referencing SPR biosensor.

FIGS. 6A and B show self-referencing for TKH2 antibody interactions with the sialyl-Tn antigen (FIG. 6A) and anti-CEA antibody interactions with the CEA antigen (FIG. 6B).

FIG. 7 illustrates the digital window system as disclosed in U.S. Pat. No. 6,747,780.

FIG. 8 is an SPR sensorgram showing detection of CEA at concentrations of 10 ng/ml, 100 ng/ml, 1 μg/ml, and 10 μg/ml.

FIG. 9 shows confirmation of specific binding between CEA and CEA-specific antibodies using BSA and non-specific mouse IgG as reference molecules (1), and the corresponding SPR sensorgram using BSA as referencing molecule (2).

FIG. 10 shows an SPR sensorgram detecting CEA at a concentration of 1 ng/ml.

### DETAILED DESCRIPTION OF THE INVENTION

The present invention provides a surface plasmon resonance (SPR) system, and corresponding methods of use, for determining the presence of antigens in patient samples, such as tumor associated antigens in serum.

The SPR system of the invention may be used to determine the presence or concentration of multiple tumor-associated antigens in a sample. Alternatively, the SPR system of the invention can provide for determination of a single antigen in a number of different samples, to thereby reduce the expense of analysis.

The invention, when determining the presence of two or more tumor-associated antigens, or when determining the presence of an antigen in multiple samples, employs an SPR system having a plurality of channels, where each channel has an antibody specific for a tumor-associated antigen operably affixed to the surface thereof A biological sample(s) is applied to the SPR system, and an SPR signal shift is measured from each of the channels to determine the presence or concentration of a tumor-associated antigen. In view of the fact that determining the presence or concentration of a single tumor-associated antigen is often insufficient for diagnosis or prognosis of disease, the SPR biosensor of the invention provides for improved methods and systems for detecting and monitoring the progression of cancer.

The SPR system and method of the invention is suitable for determining the presence and/or concentration of analytes present at very low concentrations in patient samples, for example, analytes present in the range of about 1 ng/ml to about 10 ng/ml in patient samples. Thus, the present invention allows for efficient and convenient detection of, for example, CEA in patient serum, which is typically present at about 2.5-5 ng/ml.

The invention further allows for the detection of carbohydrate antigens associated with cancer, such as the detection of carbohydrate antigens selected from Le^{x}, dimeric Le^{x}, sialyl Le^{x}, sialyl Le^{a}, sialyl Tn, Tn, disialyl Lc₄, sialyl dimeric Le^{x}, and GalNAc disialo Lc₄ (see Table 1). These antigens are associated with cancers such as cancers of the lung, breast, GI, colon and prostate (see Table 2). Suitable antibodies specific for these carbohydrate antigens are available, and are also summarized in Table 1.

Tumor-associated carbohydrate epitopes are not by themselves optimal analytes for sensitive SPR analysis. However, these antigens are also present in sera as bound to specific carrier proteins as glycoproteins or lipoglycoproteins. Thus, in a preferred embodiment of the invention, the SPR channel(s) containing antibodies specific for a tumor-associated carbohydrate antigen further have operably affixed to the surface thereof, an antibody specific for the carrier polypeptide to which the carbohydrate antigen is associated in cancer patients. Thus, the channel(s) for detecting a carbohydrate antigen preferably contain two antibodies, one directed to the polypeptide, and the other directed to the glycosyl epitope. Preferably, the antibodies bound to the sensor surface are Fab fragments. Such provides for superior detection of tumor-associated carbohydrate antigens in an SPR system.

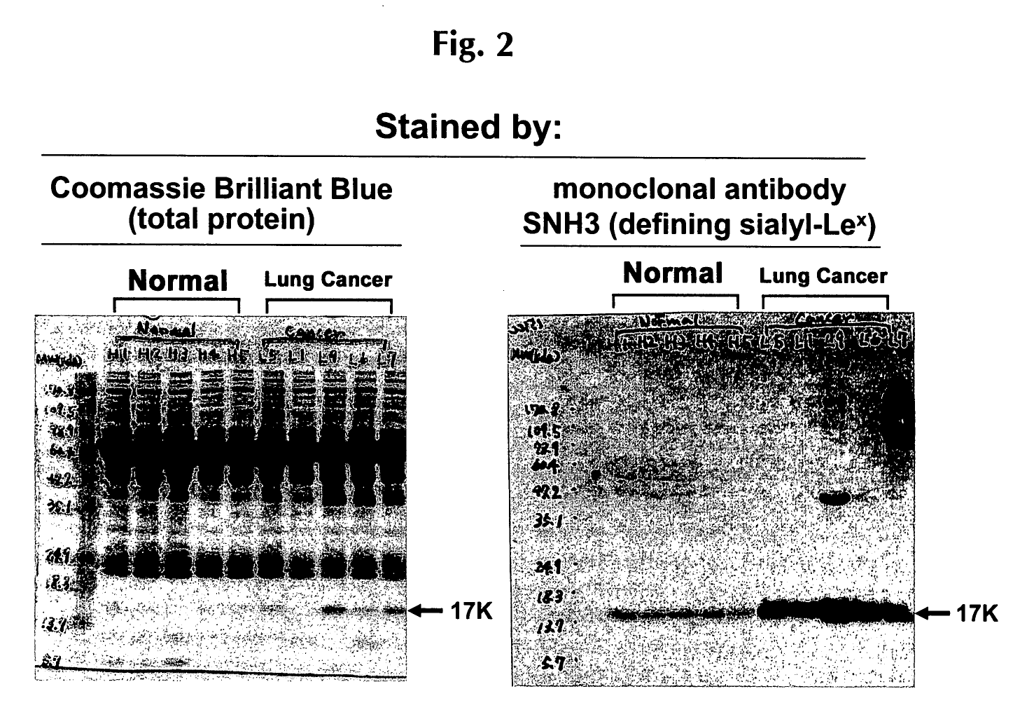

For example, in a preferred embodiment, the SPR system and method of the invention detects the presence or concentration of Sialyl-Le^{x}. Sialyl-Le^{x} is a well-established tumor-associated antigen originally identified as a glycosphingolipid (see Table 1), and is specifically observed in Western analysis of lung cancer patient sera, as a band with a molecular mass of 17 kDa (see FIG. 2). This 17 kDa protein has been identified as haptoglobin alpha 2 chain (see Table 2).

Thus, in one embodiment, the SPR system and method of the invention employs an antibody specific for the glycosyl eptitope of Sialyl-Le^{x}, such as SNH3, as well as an antibody specific for haptoglobin alpha 2 chain, to provide greatly enhanced sensitivity of detection.

An N-linked complex of fucosylated glycan linked to haptoglobin beta chain is also a good marker for diagnosing pancreatic cancer (Okuyama, et al.,Fucosylated haptoglobin is a novel marker for pancreatic cancer: a detailed analysis of the oligosaccharaide structure and a possible mechanism for fusosylation, Int. J Cancer 118:2803-2808, 2006), and thus the present invention includes an SPR system employing antibodies directed to the fucosyl epitope and haptoglobin beta chain. The structure of the fucosyl epitope, as disclosed in Okuyama, is herein incorporated by reference.

The present invention preferably uses IgG monoclonal antibodies, and more preferably monoclonal antibody fragments such as a Fab fragments, affixed to the sensor surface. IgG1, IgG2, and IgG3 subtypes are preferable (see Table 1). In the case of tumor-associated carbohydrate antibodies, many anti-carbohydrate antibodies are of the IgM isotype. Therefore, it it may be necessary to produce the corresponding IgG antibody (Fukushi Y., et al., J Biol Chem 259(16): 10511-7, 1984).

[39] In certain embodiments of the invention, the sample is first subjected to a separation means to at least partially enhance the amount of target antigen per total protein content, which can further enhance the SPR analysis since serum has a large amount and number of protein components that can potentially cause non-specific SPR changes.

The SPR system of the invention preferably employs a sensor surface having a gold substrate, with antibodies operably affixed to the sensor surface directly or through a linking layer, such as a self-assembled monolayer (SAM). Alkanethiols of 11-18 carbons in length spontaneously form stable monolayers on the surface of gold, and thus are preferred components of the SAM. Hydroxyl-terminated SAMs are also preferred to mimic protein resistance (Li et al., Langmuir 19, 3266-3271, 2002). In one embodiment, the SAM comprises 16-mercaptohexadecanoic acid or a mixture of 16-mercaptohexadecanoic acid and 11-mercaptoundecanol.

The linking layer may be a planar, two-dimensional surface, such as a self-assembled monolayer, or a three-dimensional matrix composed of, for example, dextrans (Karlsoon et al., Methods 9, 99-110, 1994). Advantages of the planar, two-dimensional surfaces include the ability to better control spatial and orientation properties by modulating the monolayer components (Bamdad C, Biophys. J 75, 1989-1996, 1998).

Various SPR configurations are known, and may be adapted for use with the invention.

In the angle-modulation mode of SPR, the wavelength of the light is held constant, and the angle of incidence is varied. In the Kretschmann configuration, a rotation stage may be used to perform the angular scans. Specifically, a sensing cell (prism, glass slide coated with metal and sensing layer, and flow cell) is mounted on a revolving table and illuminated with p-polarized, monochromatic light. A detector positioned on the outer section of the table then monitors the intensity of the reflected beam.

To minimize moving parts, a “fan-type” SPR biosensor may be employed, in which a “fan” of light illuminates a point on the metal film with a range of angles simultaneously, instead of scanning a collimated light beam with a single incident angle. The reflectivity versus angle-of-incidence profiles can thereby be obtained simultaneously. An advantage of this kind of set-up is that SPR shifts can be obtained in real-time without performing an angular scan.

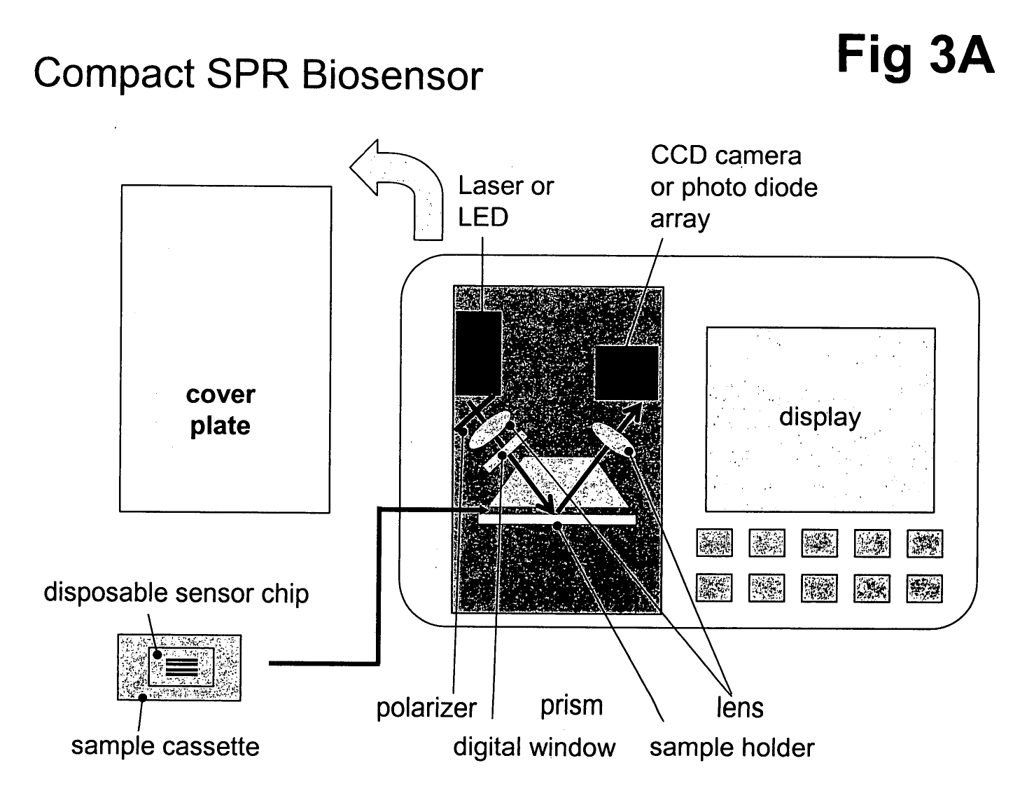

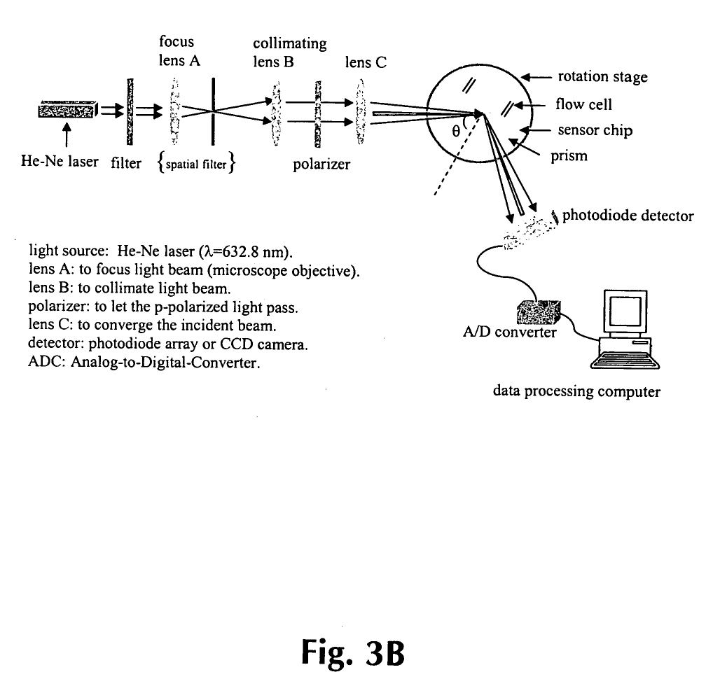

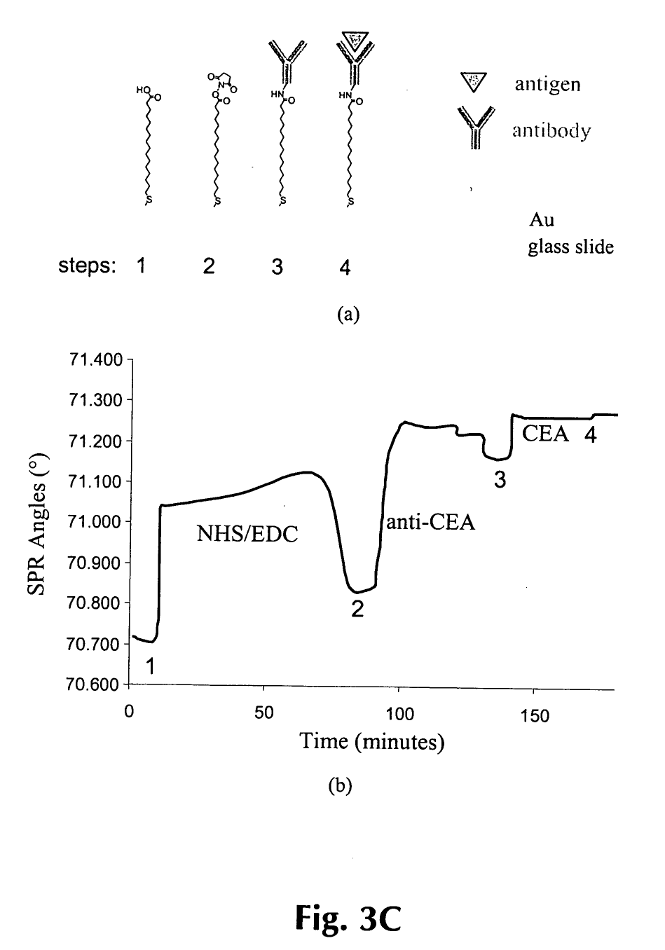

An exemplary SPR system employs a monochromatic light source such as a He-Ne laser or a laser diode, a polarizer, a lens, and a prism such as a dove-type or semi-cylindrical glass prism, as illustrated in FIG. 3.

The SPR angle, the incident angle at which the intensity of the reflected light becomes minimum, is monitored by a detector, photodiode array, or CCD camera. The intensity of reflected light vs. image pixel number may be recorded by software for the detector. Both a still image and a video may be recorded. For video, one frame may be taken per second, for example. The image pixel number may be converted to an angle through calculation, using water and 10% ethanol as standard material.

The SPR system of the invention allows for the flow of a sample, such as a patient serum sample, to the sensor surface. The sample may be applied to the sensor surface by way of a sample cassette having a place for insertion of a sensor chip (FIG. 3)

The present invention also provides a disposable unit for use in a commercially-distributed kit for the SPR system. The disposable unit is a sensor chip having the antibodies, as described herein, affixed thereto. The sensor chip may be simply inserted into a sample cassette holding a patient sample to be tested.

The disposable sensor chip may have different antigen-specific antibodies affixed to the sensing surface in each of multiple channels for the determination of multiple analytes in a single sample, or alternatively, may have the same antibody affixed to the sensing surface in all channels, for the determination of the same analyte in multiple samples.

For samples with low concentrations of analyte, the signal shift caused by the interaction of interest can be smaller than environmental drift changes. Thus, the SPR biosensor of the invention is preferably well-controlled for non-specific binding events and environmental changes, such as solution or temperature changes, which can cause some shift in the SPR signal.

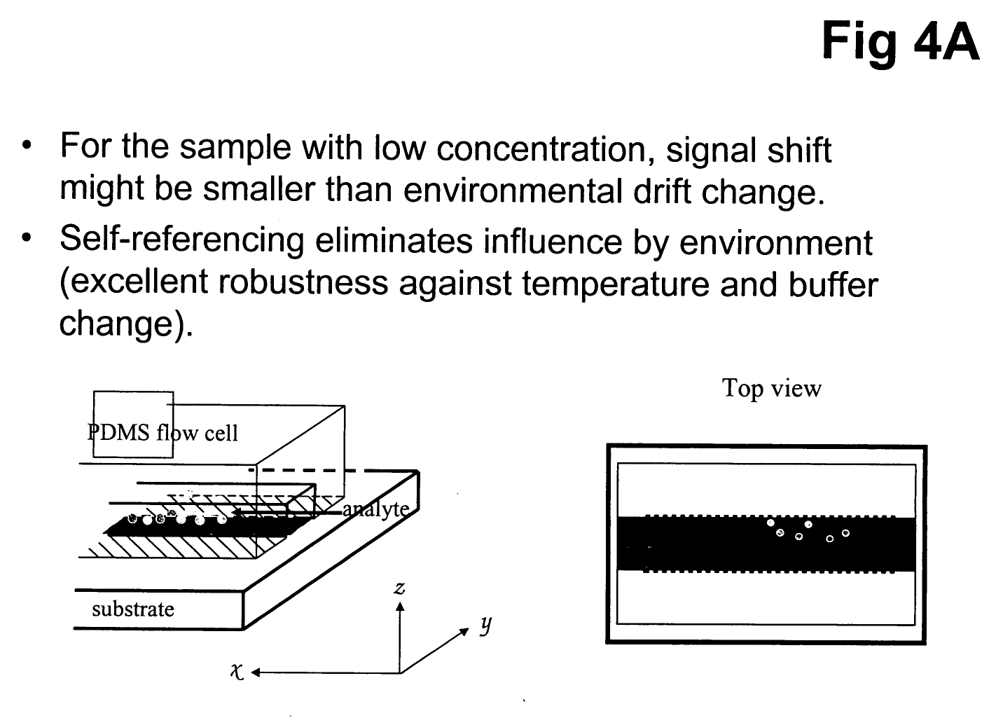

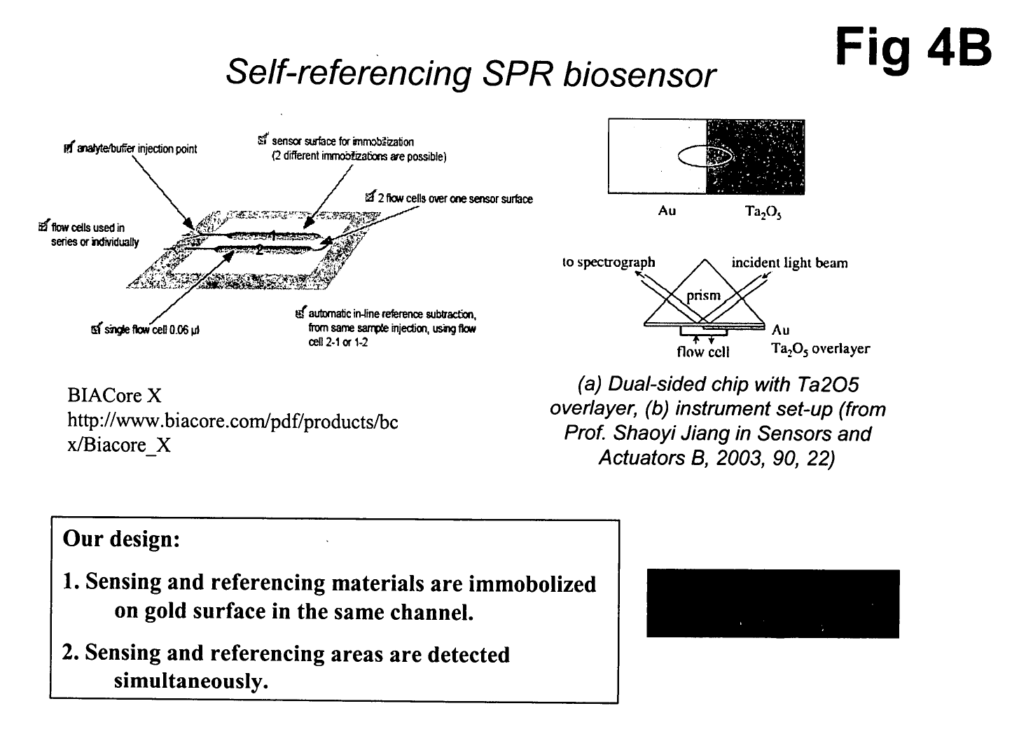

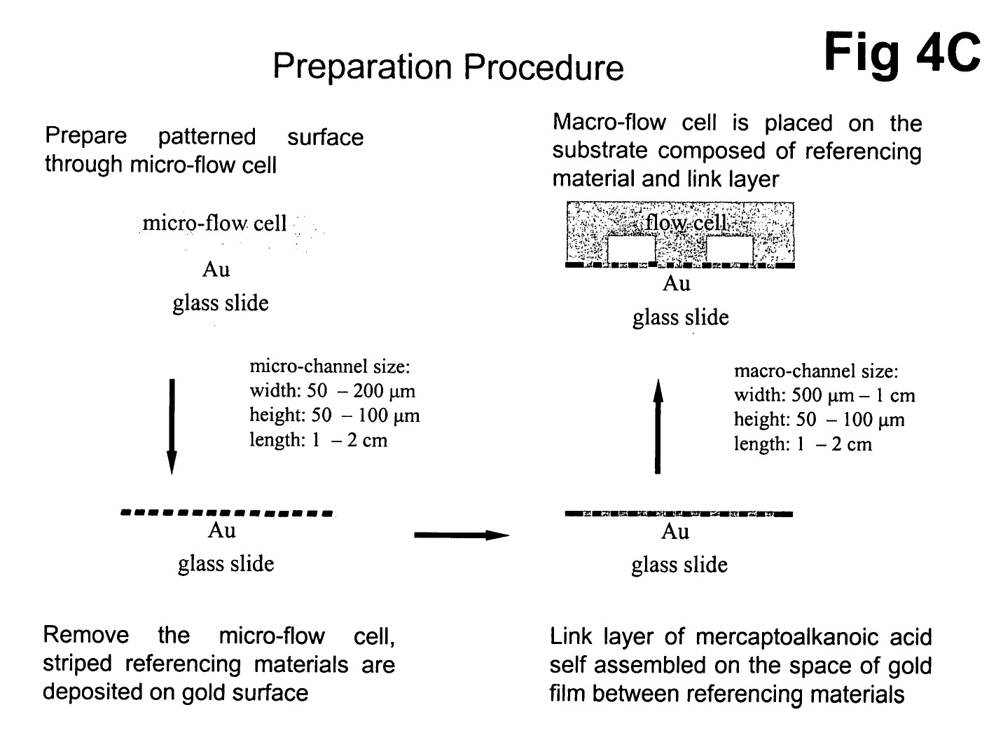

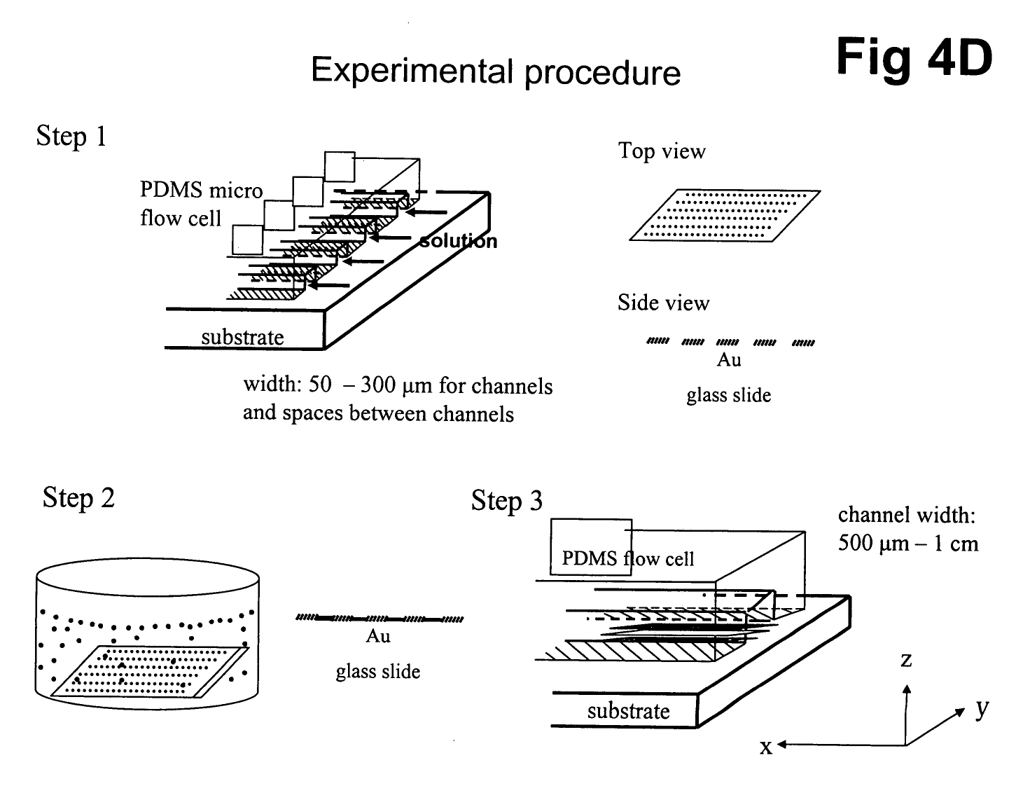

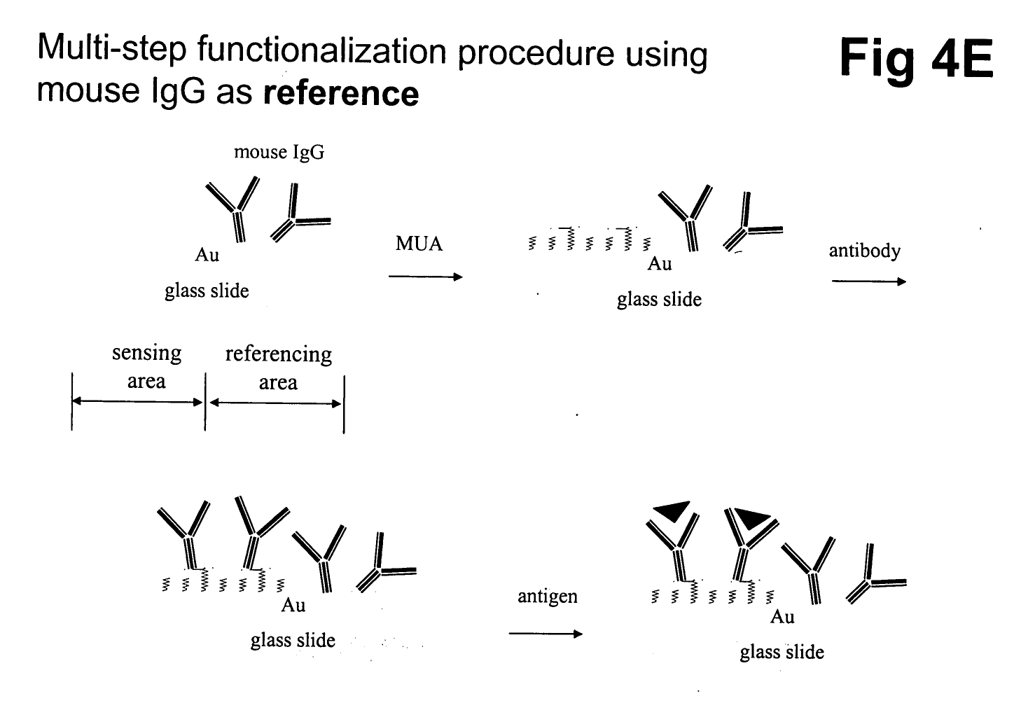

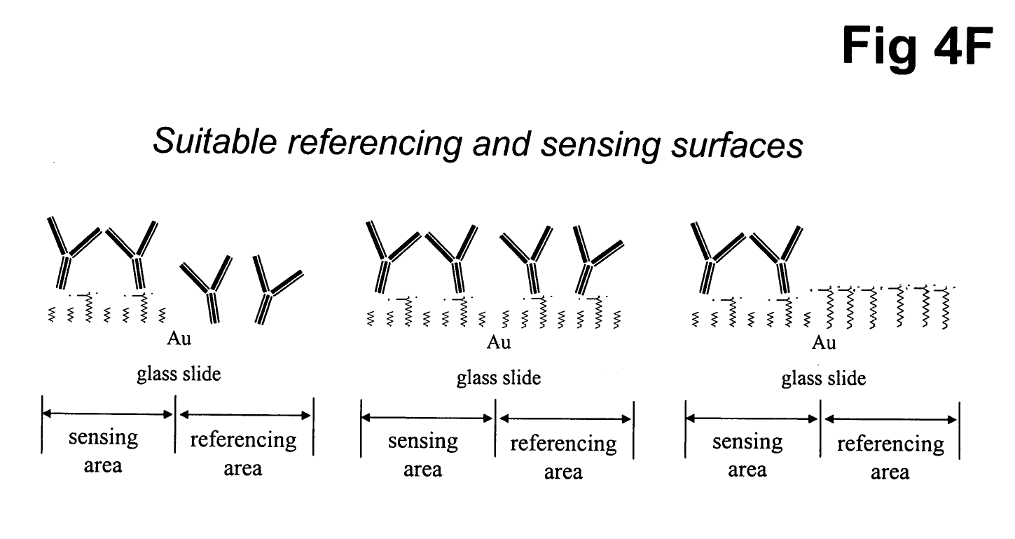

In this preferred embodiment, the SPR system is self-referencing, and is suitable for determining the presence of very low concentrations (<10 ng/ml) of antigens in patient samples. The self-referencing SPR system controls for non-specific binding reactions and environmental changes (FIG. 4).

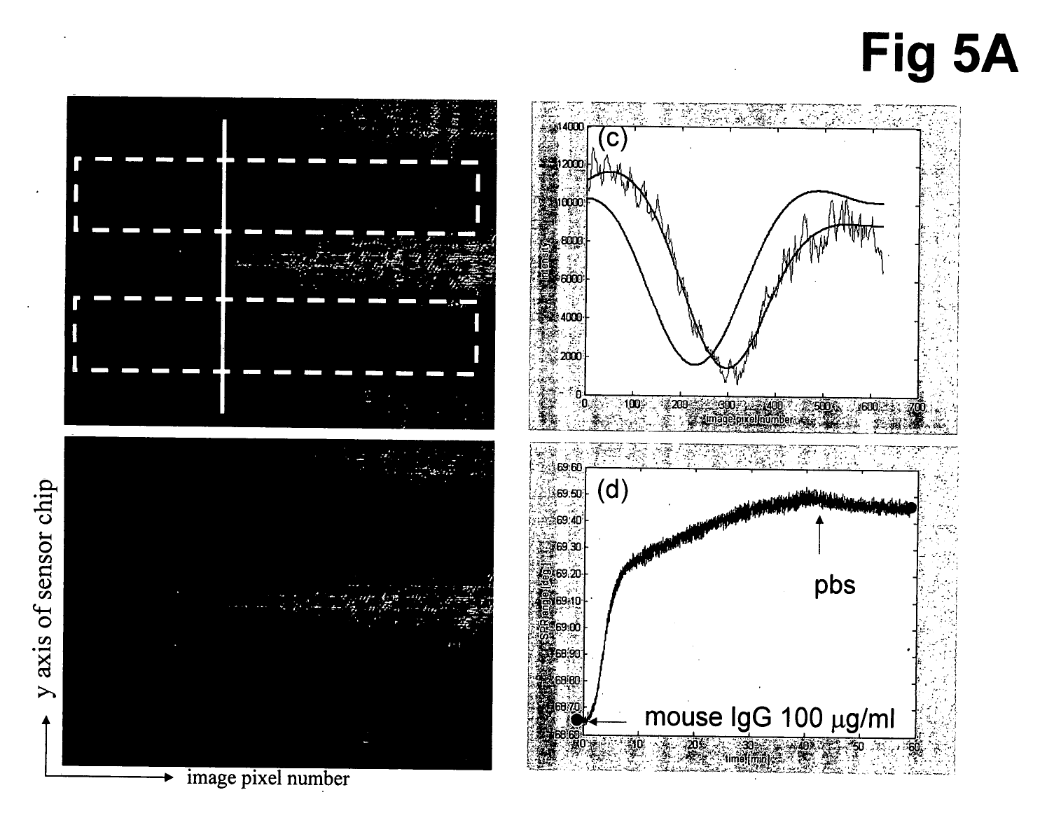

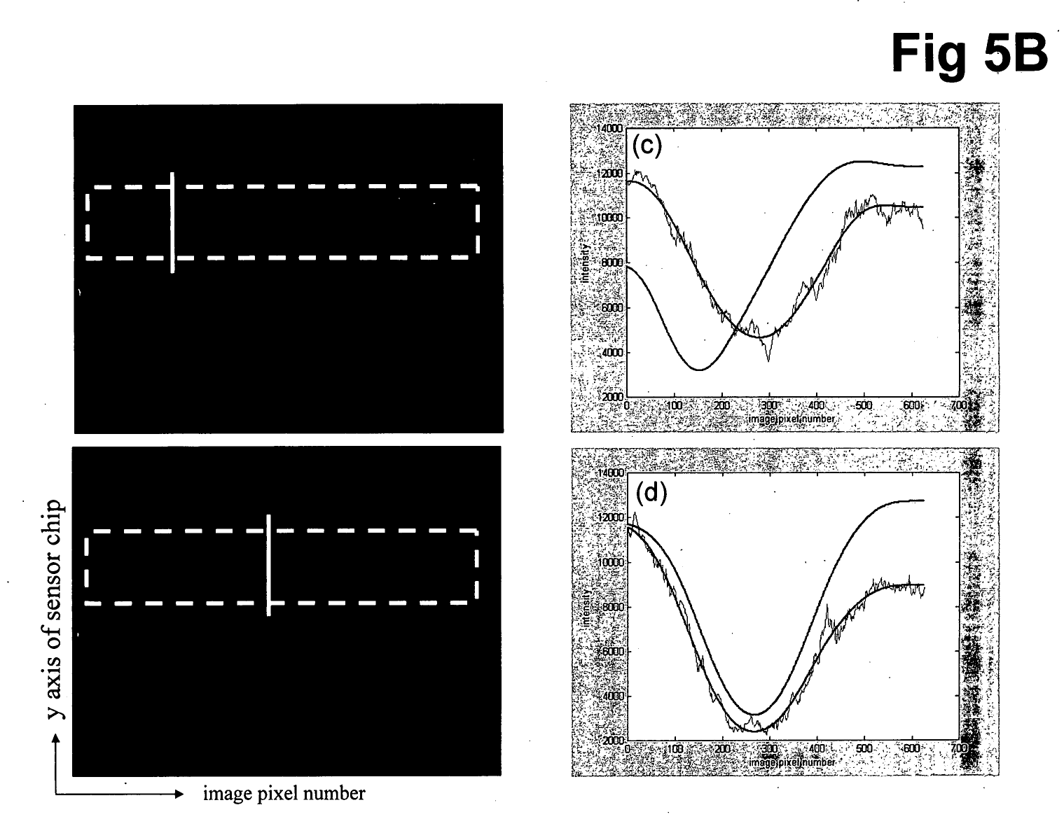

A self-referencing SPR system comprises a sensor surface with one or more channels, with each channel having a striped-patterned surface. One striped area (called “the sensing area”) is affixed with one or more antibodies against the antigen of interest, and at least one additional striped area (called “the referencing area”) allows for control of environmental changes and/or non-specific binding. For example, the referencing area may be affixed with self-referencing control antibody, such as mouse IgG (FIGS. 4 and 5) or fragment thereof. A channel may contain more than one referencing area, to allow for further controls. For example, individual referencing areas may measure the SPR signal with sensing surface alone, with unconjugated SAM, and/or with control antibody bound to the surface.

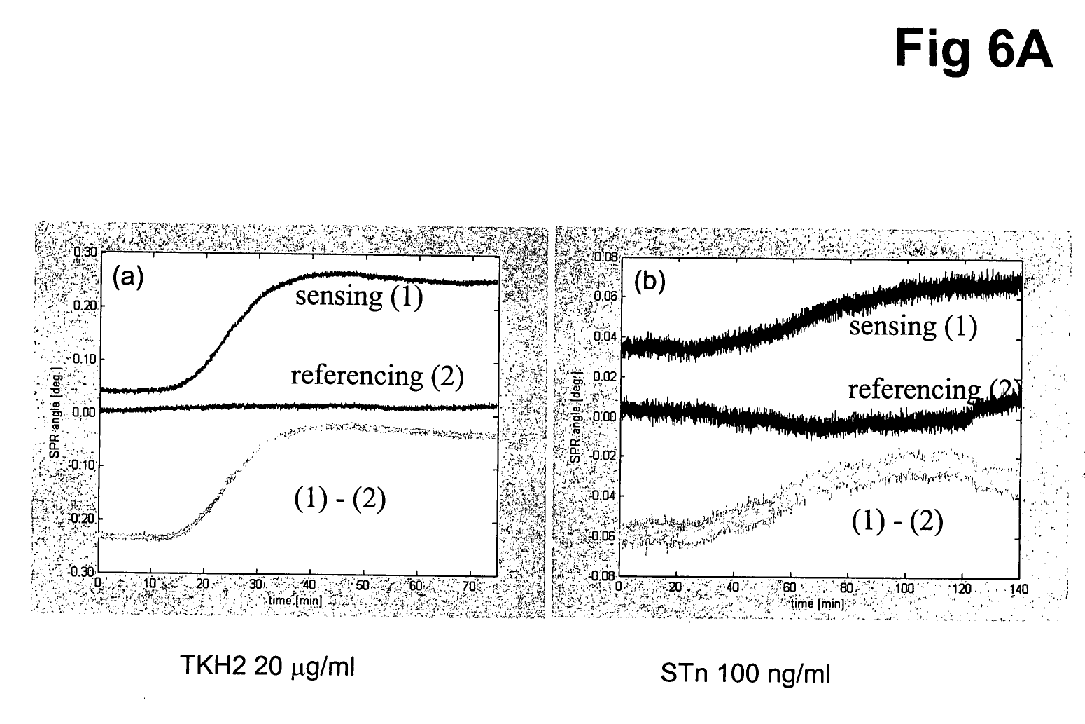

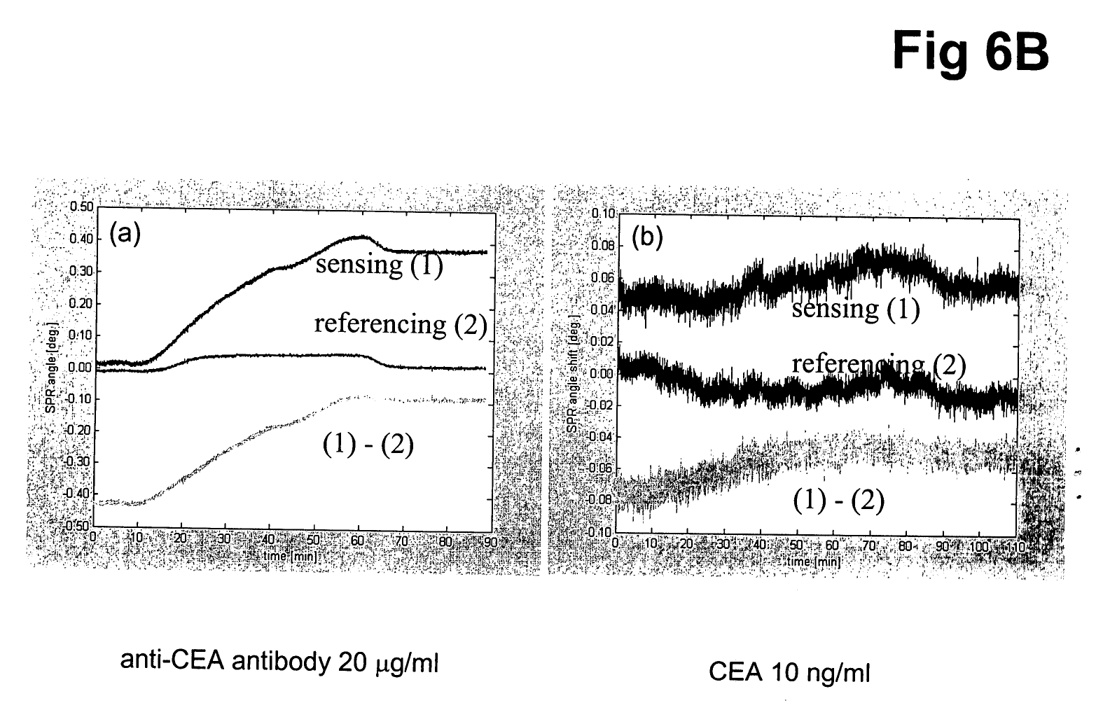

When a sample is applied to the multiple channels with a self-referencing system, and SPR signals from all of the channels are recorded sequentially, the SPR system can determine the SPR signal shift that is due specifically to the interaction of interest by subtracting the shift due to environmental and non-specific influences. Specifically, the SPR system detects an SPR signal shift on the sensing and referencing areas simultaneously, and subtracts the SPR signal shift on the referencing area(s) from the SPR signal shift on the sensing area (FIGS. 6A and 6B). Also see, U.S. Provisional Application entitled, “Design of Surface Plasmon Biosensor Based on Self-Referencing and Digital Window System,” filed Jul. 15, 2005.

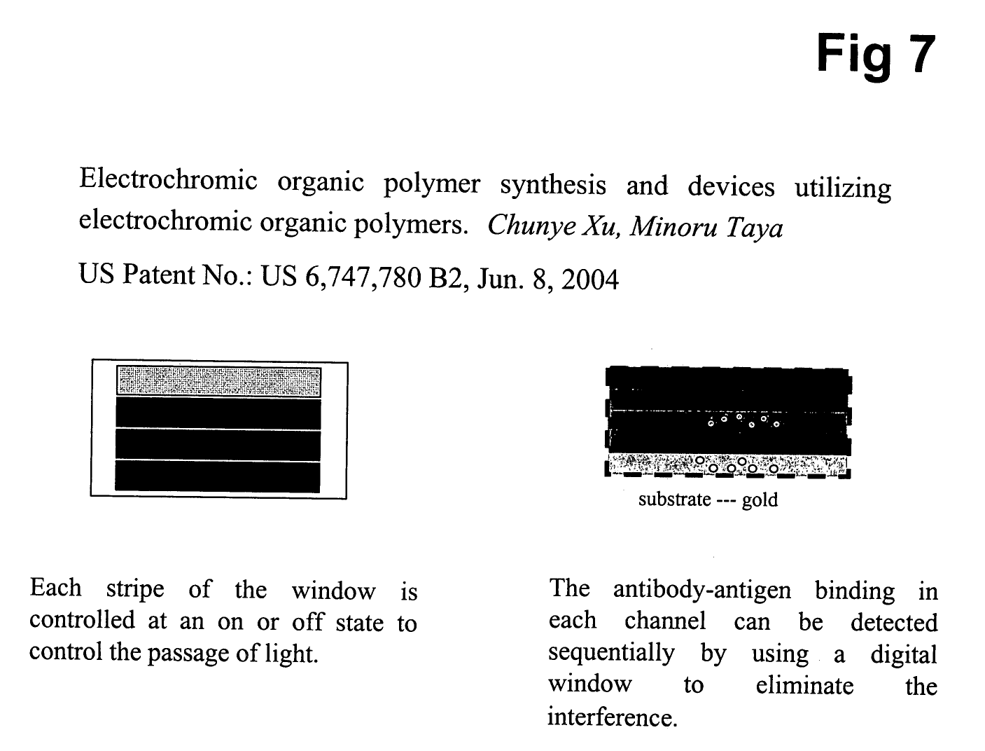

The SPR system may further comprise a digital window system placed between the light source and prism, keeping each channel in an on or off state by controlling illumination of the channels (FIG. 7).

A digital window is a patterned electrochromic (EC) material device, which is composed of a transparent electrode, a cathodic EC material that changes its color when voltage is applied, an electrolyte, and a counter-electrode. For the stripe-patterned digital window, each channel may be controlled in an on or off state by controlling the passage of light. Combined with the SPR apparatus, the antibody-antigen interactions in each channel will be detected sequentially by using the digital window to eliminate interference.

The construction and use of a digital window system has been disclosed in U.S. Pat. No. 6,747,780, the disclosure of which is herein incorporated by reference in its entirety.

### EXAMPLES

### Example 1

### Multiple Antigens Expressed in Different Loci of a Single Primary Human Tumor (“Mosaicism”)

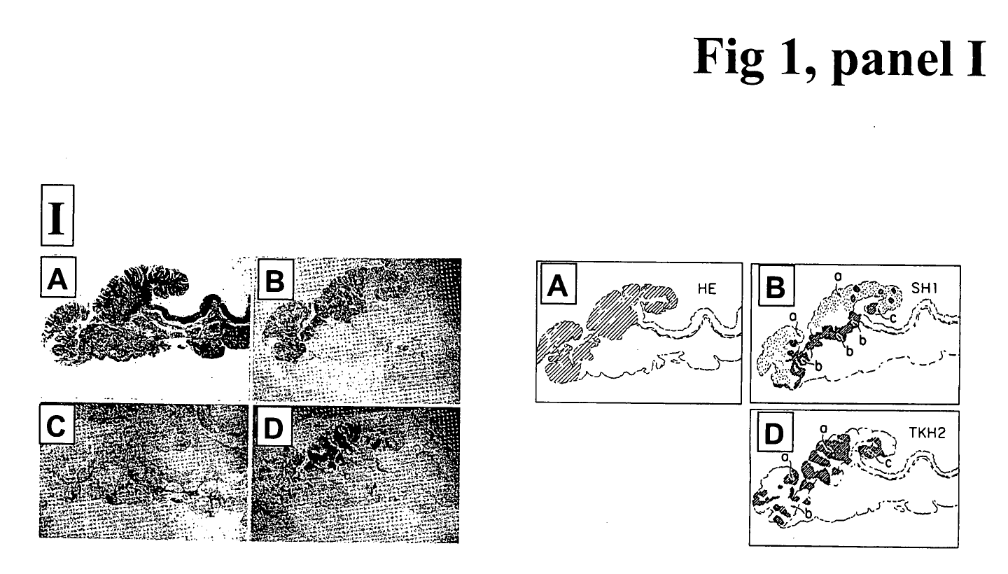

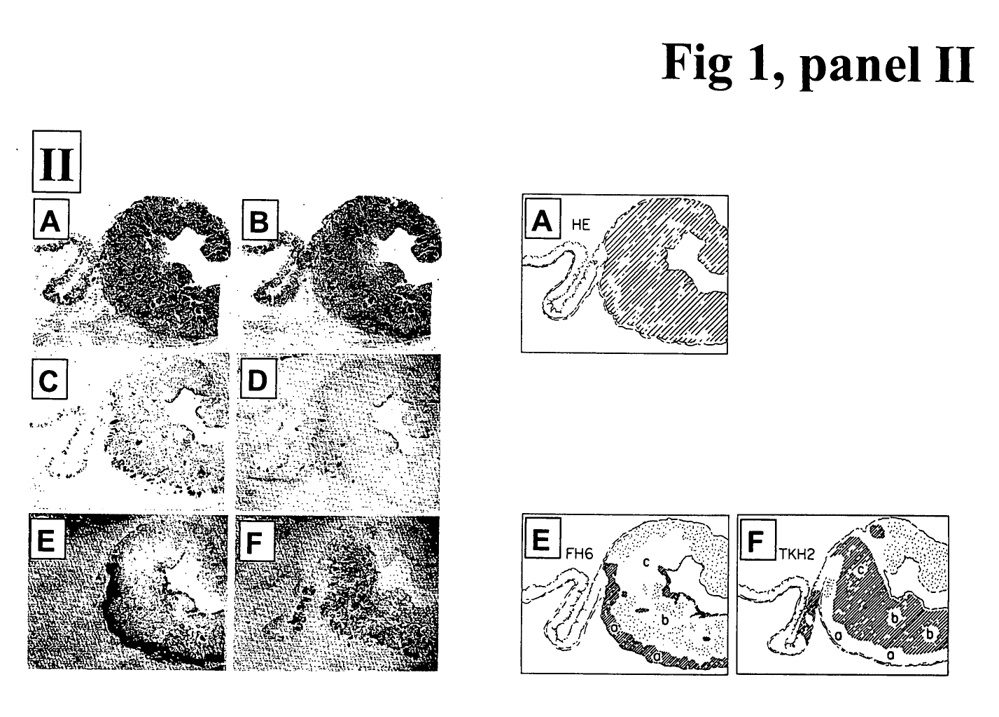

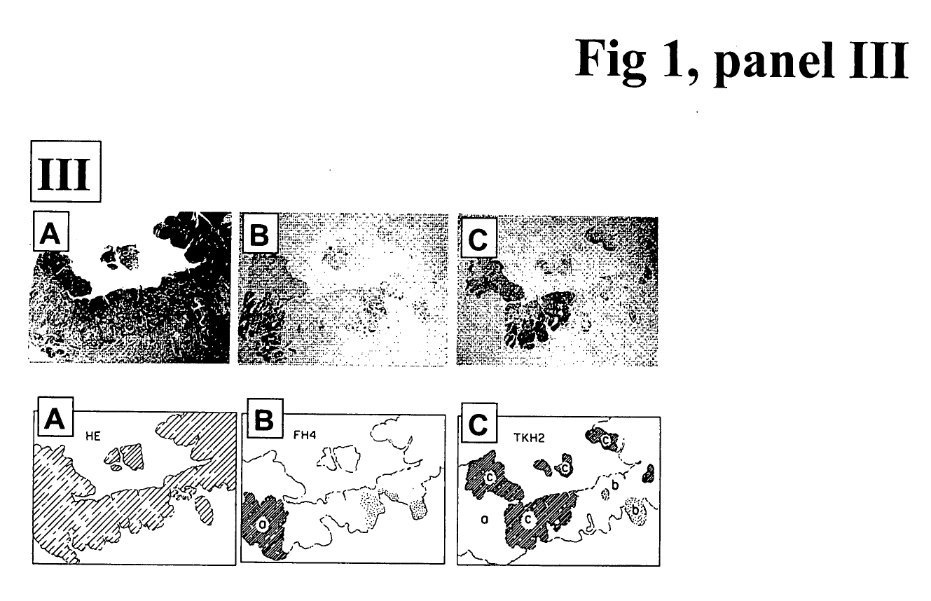

FIG. 1 demonstrates that one locus of a tumor is stained by one monoclonal antibody (mAb), a second locus is stained by a different mAb, a third locus is stained by a different mAb, etc. Such a mosaic pattern may change depending on the stage of differentiation and tumor progression. This demonstrates the importance of determining the presence of multiple antigens in a sample from a single cancer patient, making SPR analysis particularly desirable for cancer diagnosis and prognosis.

FIG. 1, Panel I. Example of primary colonic cancer. (A) Hematoxylin/eosin staining. (B) Le^{x} staining by mAb SH1. (C) Sialyl dimeric Le^{x} staining by mAb FH6. (D) Sialyl-Tn staining by mAb TKH2. The entire tumor section was stained by SH1; some areas were stained strongly (area b) and others relatively weakly (area a) (right, B). Some areas (a) weakly stained by SH1 were strongly stained by TKH2, whereas some areas (b) strongly stained by SH1 were not stained by TKH2 (left and right, D). Diffuse positive staining with sporadic strong staining at membranes with FH6 was observed (left, C).

FIG. 1, Panel II. Example of primary gastric cancer. (A) Hematoxylin/eosin staining. (B) Sialic acid staining by periodate/Schiff reagent. (C) Le^{x} staining by mAb SH1.(D) Dimeric Le^{x} staining by mAb FH4. (E) Sialyl dimeric Le^{x} staining by mAb FH6. (F) Sialyl-Tn staining by mAb TKH2. Sketches (right) show staining patterns of FH6 and TKH2, defining areas a, b, and c. The entire tumor section was strongly stained by periodate/Schiff reagent and by SH1. A clear complementarity of staining was found between FH6 and TKH2; i.e., area a (right) was strongly stained by FH6 but not stained by TKH2, whereas areas b and c (right) were strongly stained by TKH2 but not stained by FH6. There was weak, diffuse staining by FH4.

FIG. 1, Panel III. Well-differentiated gastric cancer. (A) Hematoxylin/eosin staining. (B) Dimeric Le^{x} staining by mAb FH4. (C) Sialyl-Tn staining by mAb TKH2. Sketches (bottom) show staining patterns of FH4 and TKH2, defining areas a, b, and c. Area a was strongly stained by SH1 (not shown) but also stained by FH4 (bottom, B) and FH6, whereas area c was strongly stained by TKH2 (bottom, C) but not stained by FH6, and weakly stained by SH1 (not shown).

### Example 2

### Compact SPR Biosensor System

An angle-modulated compact SPR biosensor system in Kretschmann configuration is exemplified in FIG. 3. A He-Ne laser or laser diode serves as a monochromatic light source. A polarizer permits the p-polarized light to pass through, and a lens is used to adjust the light beam. A dove-type or semi-cylindrical glass prism serves as a Kretschmann attenuated total reflection (ATR) coupler. The reflected light is focused on a high resolution photodiode array or CCD camera. A digital window is placed in the illuminating arm to permit the light beam to be shed on a certain channel on the sensor chip. A disposable sensor chip is placed in a sample cassette, and the cassette holding the sensor chip is then inserted into the sample holder. The sensor chip is attached over the prism with a refractive index matching liquid, or polymer film applied between them (sensor chip and the prism).

### Example 3

### Detection of CEA With An SPR Biosensor

The SPR system of this example is based on the angle interrogation technique, and has an angular resolution of 0.002°. As shown below, this SPR system is capable of detecting CEA at concentrations typical of early-stage cancer.

A system with a rotation stage was first set up to determine the absolute SPR angles of deionized water and buffer. Then, the lens system in the incident arm was adjusted and a “fan-type” SPR system without moving parts was developed for real-time observation of the antibody and antigen interactions. Through multi-functionalization, anti-CEA antibodies were immobilized on sensing gold film. Binding events between immobilized antibodies and antigens from solution were monitored by photodiode array and PC system.
1. Set-up of SPR optical system

An angle-modulated optical system in Kretschmann configuration is illustrated in FIG. 3B. A He-Ne laser (0.5 mW, Uniphase) serves as a monochromatic light source at a wavelength of 632.8 nm. Polarizers (Edmund) permit the p-polarized light to pass through. A spatial filter (Edmund) expands the light size. A dove-type glass prism (BK7, n_{D}=1.515, Thorlabs) serves as a Kretschmann ATR coupler. The reflected light is focused on a high resolution photodiode array (1024 pixels, Hamamatsu). The complete set-up is placed on an optical table.
2. Chemicals and Materials

Carcinoembryonic antigen (CEA) was purchased from Research Diagnostics, Inc., anti-CEA antibody was obtained from US Biological (Swampscott, MA), bovine serum albumin (BSA) and mouse IgG were from Sigma. 16-mercaptohexadecanoic acid, N-hydroxy-succinimide, N-ethyl-N′-(3-diethylamino-propyl) carbodiimide, phosphate buffered saline (PBS) and chlorotrimethylsilane were from Aldrich. Polydimethylsioxane elastomer kit (Sylgard 184; Dow corning, USA) was analytical grade. All other chemicals were commercial products of analytical-reagent grade. Deionized distilled-water (18 MΩ) was made using a Labconco water purification system.
3. Flow System

The flow system was composed of a syringe pump, a miniature flow cell and Teflon tubes. Chemically-inert and easily moldable poly(dimethylsiloxane) (PDMS) elastomer having microchannel structures acted as the flow-cell. The PDMS flow cell was attached to the gold surface of the sensor chip so that solutions could be easily passed through for reactions on the sensor surface.

Photolithography work for preparation of the PDMS flow cell and E-beam evaporation of metal layers was carried out in the Washington Technology Center located at the University of Washington (UW). Briefly, PDMS microchannels were processed by replication from 3-D silicon wafer masters which were made photolithographically from a 2-D Mylar mask pattern. The Mylar masks were printed at the UW Publication Service Center. The masks contained a 2-D pattern of parallel channels (width 500 μm and length 2.0 cm) and circular reservoirs (diameter 1.5 mm) at both ends of each channel. The 3-D patterns on Si wafer were made with a negative photoresist (SU-8 50, MicroChem Corp) that was spin-coated at 3000 rpm for 30 s and then exposed to 365-nm UV light (ABM Aligner). Replicas were formed from a 1:10 mixture of PDMS curing agent and prepolymer (Sylgard 184, Dow Corning) that was degassed under vacuum and then poured onto the master to create a layer with a thickness of about 3-5 mm. Before the pouring of PDMS onto the 3-D Si master, a few drops of chlorotrimethylsilane were placed around the master for several minutes to ensure easy removal of the PDMS replicas. The PDMS was then cured for 24 h. at room temperature before it was removed from the Si wafer. Reservoirs were created by cutting out the circular ends of each channel from the PDMS with a hole punch.
4. Design and Preparation of Sensor Chip

A thin gold film evaporated on a glass plate was used as the base for the SPR sensor chip. Microscope glass plates (25 mm×75 mm×1.0 mm) were used as substrates for the thin gold film. The glass slides were flushed with water and ethanol, and thoroughly cleaned by immersing them in piranha solution (30% H₂O₂, 70% H₂SO₄) for two hours. The slides were then rinsed with DI water, absolute grade ethanol, and blown dry with a stream of nitrogen before mounting them onto a rotating carousel in a vacuum chamber for electron beam metal deposition. An adhesion layer of 3 nm of chromium was deposited on the glass slides first, and then, 50 nm of gold was deposited over the chromium layer. Metal depositions were conducted at a reduced pressure of ca. 1×10⁻⁶ Torr, and the thickness of the metal depositions were monitored with a quartz balancer (CHA 600 E-beam evaporator).

The functionalization of the gold surface includes three steps as shown in FIG. 3C. First, a monolayer of 16-mercaptohexadecanoic acid (MHA) was self-assembled on the gold film. The glass slides coated with gold film were placed in a 1 mM solution of MHA in ethanol for 24 hours to form a self-assembled monolayer, and then rinsed with DI water and ethanol to remove excess and weakly bound molecules. Second, the carbonyl group of MHA was activated by a solution of 20 mM N-hydroxysuccinimide (NHS) and 50 mM N-ethyl-N′-(dimethylaminopropyl)-carbodiimide (EDC). Anti-CEA antibody was then immobilized on the self-assembled monolayer of MHA by primary amine coupling. The terminal carboxylic groups of the 16-mercaptohexadecanoic acid SAMs (step 1 in FIG. 3C) were converted to reactive anhydride groups (step 2 in FIG. 3C) that later reacted with the primary amine of the antibody (step 3 in FIG. 3C).

The profile of a typical immobilization reaction is observed as SPR sensogram (angle-time relation) as shown in panel (b) of FIG. 3C. The injection procedure is as follows: (a) EDC/NHS for 60 min; (b) PBS buffer for 10 min; (c) 20 jig/ml CEA antibodies in PBS buffer for 30 min; (d) 1 M ethanolamine pH 8.5 for 10 min; (e) 20 mM HCI for 10 min; (f) PBS buffer for 10 min; (g) 10 μ/ml CEA antigens in PBS buffer for 30 min; (h) PBS buffer for 10 min. 10 mM PBS pH 7.4 was used as the carrier solution.
5. SPR measurement procedures

The entire SPR biosensor assembly comprising an optical system, flow system, sensor chip and data analysis system is shown in FIG. 3B. A refractive index matching liquid was applied on the dove-type prism and the SPR sensor chip was placed over the prism. The flow of analyte solutions was at a flow rate of 50 μl/min or 5 μl/min using the syringe pump. Room temperature was maintained at 20° C.

The SPR angle, the incident angle at which the intensity of the reflected light becomes minimum, was monitored by photodiode array. The intensity of reflected light was recorded by the software built into the detector, and then the data were processed by using programs edited by MATLAB.
6. Detection of CEA

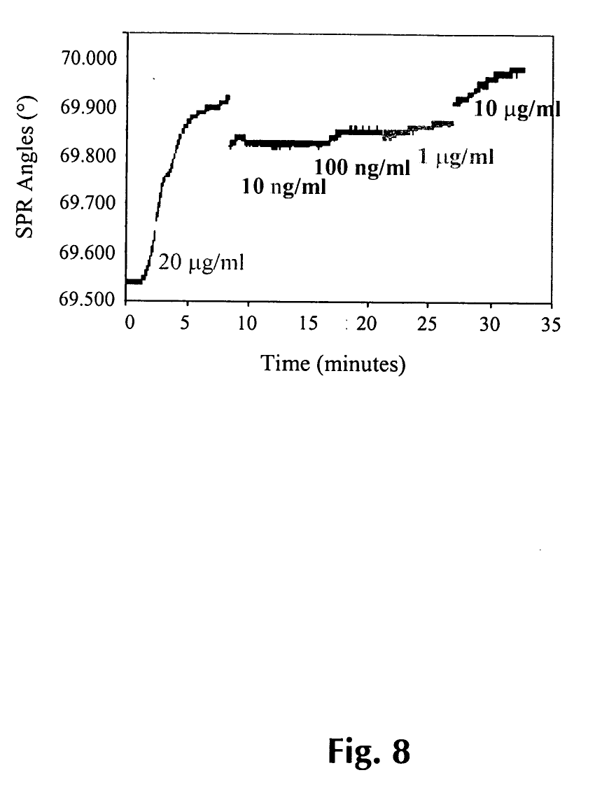

A series of concentrations of carcinoembryonic antigen (CEA) was flowed over the sensor surface prepared as above. The SPR angle was recorded as a function of time, and is shown in FIG. 8. With the increase in concentration of CEA, the SPR angle shifted to a higher degree. The SPR angle shifts of 0.017°, 0260°, 0.038° and 0.077° correspond to the concentrations of 10 ng/ml, 100 ng/ml, 1 μg/ml and 10 μg/ml of CEA, respectively.

In order to confirm that the signal shifts were due to specific antibody-antigen interactions, BSA and mouse IgG were employed as referencing reagents.

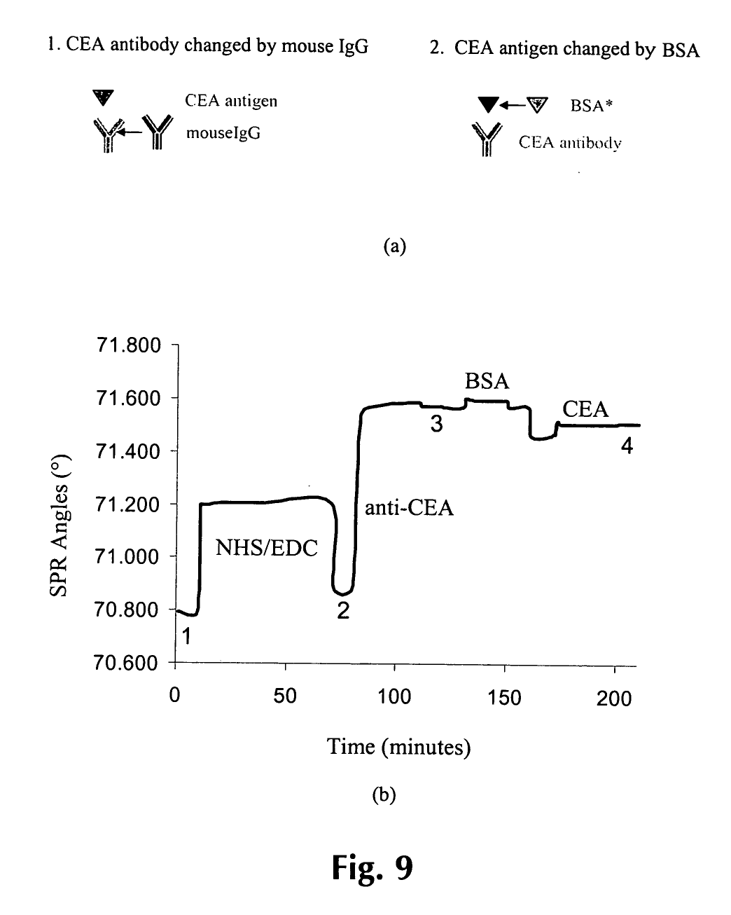

When a solution of 100 ng/ml of CEA antigen was introduced to the sensor channel, no SPR angle shift was observed on the surface having immobilized mouse IgG, compared with 0.017° angle shift for the surface of anti-CEA antibody. FIG. 9 shows the results of a second referencing experiment employing BSA. After running pure PBS buffer, a 10 ng/ml solution of BSA was introduced to test for non-specific adsorption. A small shift was observed due to the refractive index change, but after rinsing with pure PBS buffer, the signal returns to baseline. Thus, BSA does not bind to CEA antibody. When a 10 ng/ml solution of CEA was introduced to the same surface, a shift of 0.025° was observed and the signal was stable after the rinsing with PBS buffer.
7. The Detection of CEA at a Concentration of 1ng/ml

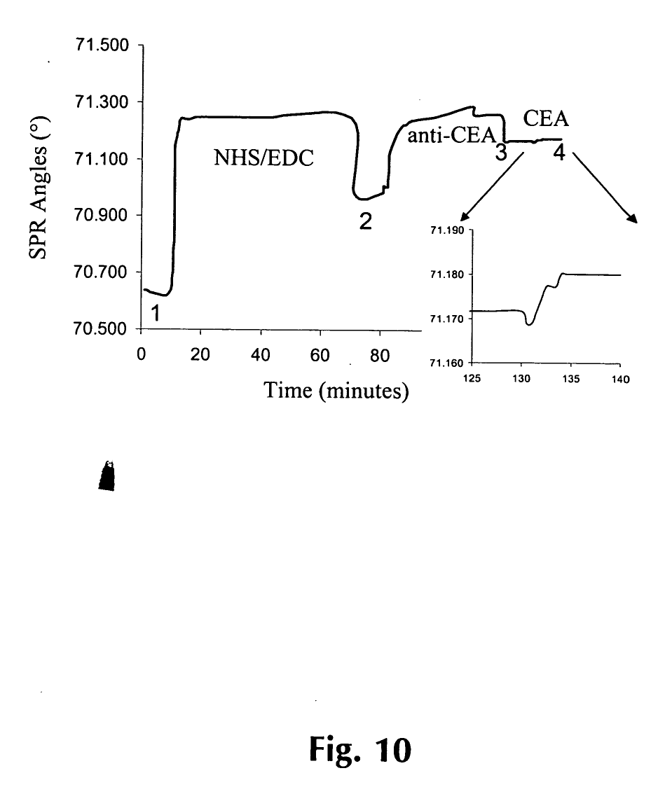

Since the normal range for CEA in an adult non-smoker is <2.5 ng/ml and <5.0 ng/ml for a smoker, detection of CEA at a concentration of around 2.5 ng/ml is required. Using the same procedure described above, CEA was detected at a concentration of 1 ng/ml. The results are shown in FIG. 10 where a 0.008° angle shift was observed.

### Example 3

### Basic Concept and Assembly of Self-Referencing SPR System

As illustrated in FIG. 3, a SPR biosensor monitors the refractive index change due to the interaction between a ligand and corresponding analyte, such as antibody and antigen. The refractive index changes cause a shift in the SPR signal. However, non-specific binding or environmental change, like solution or temperature change, can also cause a shift in the SPR signal. For samples with low concentrations of antigen, signal shift might be smaller than environmental drift change. Accurate referencing in an SPR biosensor can eliminate environmental influences.

FIG. 4B, illustrates the self-referencing BIACore X biosensor and the dual-sided chip with a Ta₂O₅ overlayer disclosed in C. Boozer et al., Surface functionalization for self-referencing surface plasmon resonance (SPR) biosensors by multi-step assembly, Sensors and Actuators B 90:22-30 (2003), which is hereby incorporated by reference in its entirety.

In the BIACore X SPR biosensor, one channel is used as the referencing channel, and the other as the sensing channel. However, with greater sensitivity comes greater interference from environmental factors. Thus, a referencing surface together with a sensing surface in one channel is preferable to allow the SPR signal from both the sensing and referencing surfaces to be determined simultaneously, and under the same environmental conditions.

A wavelength-modulated self-referencing SPR biosensor, in which gold is used as the sensing surface, while a Ta₂O₅ overlayer is used as the referencing surface, may be used. However, deposition of Ta₂O₅ on gold takes some time and effort, and thus it is preferable to employ gold as substrate for both sensing and referencing areas, which is quicker and easier to prepare.

FIG. 4C illustrates the placement of micro and macro-flow cells on a gold-coated glass substrate and emphasizes the difference between micro vs. macro-flow cell dimensions (dimensions shown may vary), and the change on the gold surface from a side view.

FIG. 4D illustrates steps in the preparation of an exemplified SPR system of the invention: (1) preparation of a stripe-patterned referencing surface through micro-flow cells; (2) formation of a self-assembled monolayer on the exposed gold surface between referencing materials; (3) change to macro-flow channel, for the test of antibody/antigen interaction.

FIG. 4E illustrates the sensing and referencing material structure, with mouse IgG on the referencing surface, and anti-tumor antibodies on the sensing surface, to determine antigen-antibody binding from simultaneous SPR signal shifts from the sensing surface and the referencing surface.

FIG. 4F, illustrates examples of referencing and sensing surfaces. The antibodies may be absorbed onto the gold surface directly or through a linking layer, such as oligo(ethylene oxide) terminated alkanethiols. Alternatively, antibodies may be immobilized onto a linking layer of mercaptoalkanoic acid. The referencing surface in one embodiment may consist only of the linking layer.

### Example 4

### Fabrication of Sensor Chip and Preparation of Sensing Surface

Sensor chips with sensing and referencing areas in a striped pattern are aligned with polydimethylsiloxane (PDMS) flow cells containing multiple channels. In each channel, a different IgG mAb (as sensing material), and mouse IgG (as referencing material) is affixed to a gold surface directly or through SAM. Exemplary IgG mAbs directed to a tumor-associated antigen are anti-Tn (CU1; IgG3), anti-sialyl-Tn (TKH2; IgG1), anti-sialyl-Le^{x} (SNH3; IgG3), anti-sialyl-Le^{a} (NKH1; IgG1), or anti-disialyl-Lc4 (FH9; IgG2a) (Table 1).

Microfluidic channels were fabricated in a poly(dimethylsiloxane) (PDMS) polymer. Briefly, PDMS microchannels were created by replication from 3-D silicon wafer masters that were created photolithographically from a 2-D Mylar mask pattern.

Mylar masks were printed at the University of Washington Publication Service Center. The masks contained a 2-D pattern of parallel channels (width 500 μm and length 2.0 cm) featuring circular reservoirs at both ends of each channel.

The 3-D patterns on a Si wafer were made with a negative photoresist (SU-8 50, microlithography Chemical Corp., Newton, Mass.) that was spin-coated at 5000 rpm for 20 s and then exposed to 365-nm UV light. The 3-D silicon master was silanized by a few drops of chlorotrimethylsilane (Sigma-Aldrich, St. Louis, Mo.), which ensures the easy removal of the PDMS replicas from the Si master.

Replicas were formed from a 1:10 mixture of a PDMS curing agent and a prepolymer (Sylgard 184, Dow Corning, Midland, Mich.) that was degassed under vacuum and then poured onto the master to create a layer with a thickness of about 0.5-1 mm. The PDMS was then cured for at least 1 h at 70° C. before it was removed from the Si wafer. Reservoirs were created by cutting out the circular ends of each channel from the PDMS with a hole punch.

A sensing surface of the SPR system of the invention may be prepared as follows.

100 μg/ml solution of mouse IgG in PBS is allowed to flow through 200 μm microchannels of PDMS at a flow rate of 0.01 ml/min. After the PDMS is removed, stripes of mouse IgG deposited on the gold surface can be observed.

A monolayer of ω-mercapto aliphatic acid is self-assembled on a gold film. For example, a mixture of 16-mercaptohexadecanoic acid (MHA) and 11-mercaptoundecanol (MUO) may be used. Glass slides coated with gold film are placed in a 1 mM solution of MHA and MUO in a molar ratio of 1 to 9 in ethanol for 24 hr to form a self-assembled monolayer, and then rinsed with deionized water and ethanol to remove excess and weakly bound molecules.

The carboxyl group of MHA is activated by a solution of N-hydroxysuccinimide (NHS) and N-ethyl-N′-(dimethylaminopropyl)-carbodiimide (EDC). Then, antibody is immobilized on the self-assembled monolayer of the MHA by primary amine coupling. When analyte solutions flow through the sensor surface, specific antigen-antibody interactions occur, resulting in a change of the SPR signal.

### Example 5

### Determination of Sialyl-Tn Antigen

a. Preparation of gold film on glass slide as substrate. A thin gold film evaporated on glass plate was used as a base for an SPR sensor chip. Microscope glass plates (25 mm×37.5 mm×1.0 mm) were used as substrates for the thin gold film. The glass slides were flushed by water and ethanol first, and thoroughly cleaned by immersing them in “piranha solution” (30% H₂O₂, 70% H₂SO₄) for two hours, then rinsed with DI water, absolute grade ethanol, and blown dry with a stream of nitrogen before mounting them onto a rotating carousel in a vacuum chamber for electron beam metal deposition. An adhesion layer of 3 nm of chromium was deposited on the glass slides first, and then, 50 nm of gold was deposited over the chromium layer. Metal depositions were conducted at a reduced pressure of approximately 1×10⁻⁶ Torr, and the thicknesses of metal depositions were monitored with a quartz balancer (CHA 600 E-beam evaporator).

b. Adsorption of mouse IgG as referencing materials in a striped pattern on gold surface. In order to conduct a self-referencing experiment, referencing and sensing materials were immobilized onto a gold surface in a striped pattern. In this example, mouse IgG was used as the referencing material, and a micro-flow cell patterning method was used to obtain a striped pattern of mouse IgG on the gold surface. A PDMS micro-flow cell with a group of 200 μm wide, 50 μm thick and 2 cm long channels was placed on the gold film, 100 μ/ml mouse IgG in phosphate buffer solution (PBS) was injected and passed through the channel at a flow rate of 0.01 ml/min. The SPR angle shift was observed as shown in FIG. 5A.

FIG. 5A, shows patterning of mouse IgG on a Au surface through 200 μm microchannels. In (a), the vertical bar indicates the SPR angle before mouse IgG was injected. In (b), the vertical bar indicates the SPR angle after mouse IgG was adsorbed onto the gold surface in the images. In (c), the curve of intensity versus image pixel number is shown. The curves correspond to before and after mouse IgG adsorption. In (d), the sensorgram of SPR angle shift with time is shown.

After mouse IgG was absorbed onto the gold surface, the PDMS micro-flow cell was removed, and the sensor chip was then washed with water and ethanol.

c. Functionalization of the sensing area on gold surface. The functionalization of the sensing gold surface included two steps. First, a monolayer of 16-mercaptohexadecanoic acid (MHA) was self-assembled on a gold film. The sensor chip was placed in a 1 mM solution of 16-mercaptohexadecanoic acid (MHA) and 11-mercaptoundecanol (MUO) (a mole ratio of 1:9) in ethanol for 24 hours to form a self-assembled monolayer on the space between mouse IgG on the gold film as shown in step 2 of FIG. 3C, and then rinsed with DI water and ethanol to remove excess and weakly bound molecules. Second, the carbonyl group of MHA was activated with a solution of 0.05 M N-hydroxysuccinimide (NHS) and 0.2 M N-ethyl-N′-(dimethylaminopropyl)-carbodiimide (EDC) for 1 hour. The second step was carried out in the PDMS macro-flow cell with a width of 1 cm, the flow rate of NHS/EDC solution was 0.01 ml/min.

d. Immobilization of TKH2 antibody on linking layer. TKH2 antibody (IgG3, see Table 1) was immobilized on the self-assembled monolayer of MHA through primary amine coupling. 20 μg/ml solution of TKH2 antibody in PBS was injected into the flow cell at a flow rate of 0.01 ml/min. PBS was injected into the channel before and after antibody injection as running buffer at the same flow rate of 0.01 ml/min. FIG. 6A shows the SPR angle shift with time when TKH2 antibody passed through the sensor chip, an SPR angle shift of 0.210 degree was observed on the sensing area, while almost no shift was observed on the referencing area.

Also see FIG. 5B, which shows the adsorption of TKH2 antibody on a patterned surface. In (a) and (b), measurements from sensing and referencing areas are shown, respectively: (a) before antibody injection, (b) after antibody adsorption. (c) shows SPR curves in the sensing area before and after antibody injection. (d) shows the SPR curves in the referencing area before and after antibody injection. There is a large shift in the sensing area, but no obvious shift in the referencing area.

Detection of sialyl-Tn antigen. Analyte solutions containing 100 ng/ml of sialyl-Tn antigen in PBS were prepared, and were allowed to flow over the sensor surface having immobilized TKH2 antibody as above. A high level of tumor-associated antigen sialyl-Tn is known to be present in ovine submaxillary mucin (Kjeldsen et al, Cancer Res 48:2214-20 1988). This mucin was prepared from ovine submaxillary gland as described previously (Hill HD, et al., J Biol. Chem. 252: 3791-8, 1977). When the analyte containing sialyl-Tn antigen was allowed to flow over the sensor surface, the signal of the SPR angle was recorded as a function of time as shown in FIG. 6A(b). By subtracting the angle shift in referencing area (2) from the angle shift in sensing area (1), the pure signal change due to the specific antigen-antibody interactions resulted in a change of SPR angle of 0.035 degree.

### Example 6

### Determination of Carcinoembryonic Antigen (CEA)

Procedures a to c for preparing the referencing pattern and for functionalizing the sensing area were the same as in Example 5.

d. Immobilization of anti-CEA antibody on linking layer. Anti-CEA antibody was immobilized on the self-assembled monolayer of MHA through primary amine coupling. 20 μg/ml solution of anti-CEA antibody in PBS was injected into the flow cell at a flow rate of 0.01 ml/min. PBS is injected into the channel before and after antibody injection as running buffer at the same flow rate of 0.01 ml/min. FIG. 6B(a) shows the SPR angle shift with time when anti-CEA antibody passed through the sensor chip, an SPR angle shift of 0.380 degree was observed on the sensing area, while almost no shift was observed on the referencing area.

e. Detection of carcinoembryonic antigens (CEA). Analyte solutions of 10 ng/ml of CEA in PBS were allowed to flow over the sensor surface immobilized with antibodies. The signal of the SPR angle was recorded as a function of time as shown in FIG. 6B(b). By subtracting the angle shift in the referencing area (2) from the angle shift in sensing area (1), the pure signal change due to the specific antigen-antibody interactions resulted in a change of SPR angle of 0.034 degree.

### Example 7

### Western Blot Analysis of Sialyl-L^{x} Antigen in Serum Samples from Normal Subjects and Cancer Patients

Serum containing 25 micrograms protein was subjected to SDS polyacrylamide gel electrophoresis using standard reference proteins with various molecular mass. This was followed by Western blot analysis using PVDF membrane. Transferred proteins on membrane were determined by: (i) Coomassie Brilliant Blue staining (FIG. 2, left panel), and (ii) staining by anti-sialyl-Le^{x} monoclonal antibody SNH3 (FIG. 2, right panel). A band having a molecular mass 17 kDa was strongly stained by SNH3 in sera from lung cancer patients, while the same band in sera from normal subjects was not stained or only faintly stained by SNH3.All other protein bands were essentially the same between sera cancer patients compared to normal subjects.

### Example 8

### Tumor-Associated Glycosyl Epitopes Associated with Hagtoglobin Alpha and Beta Chains

Western blot analysis was performed with sera from normal subjects and patients with various types of cancer, at different stages, using monoclonal antibodies defining tumor-associated antigens. L, lung cancer. B, breast cancer. G, gastric cancer. C, colon cancer. M, prostate cancer. Only haptoglobin alpha 2 chain with molecular mass 17 kDa was strongly associated with anti-sialyl-Le^{x} monoclonal antibody SNH3 staining.

All patents, articles and other references cited herein are incorporated by reference in their entireties.

TABLE 1
Important mAbs directed to tumor-associated carbohydrate
antigens detectable in sera of patients with cancer.

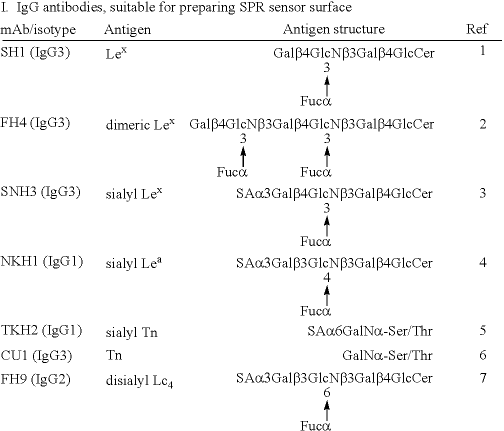

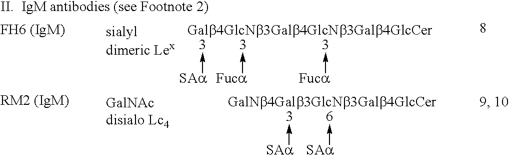

References
1. Singhal AK, et al. Cancer Res 47: 5566-71 (1987).
2. Fukushi Y, et al. JBC 259: 4681-5 (1984).
3. Phillips ML, et al. Science 250: 1130-2 (1990).
4. Hakomori S. U.S. Pat. Nos. 5,389,530 and 5,500,215.
5. Kjeldsen T, et al. Cancer Res 48: 2214-20 (1988).
6. Takahashi HK, et al. Cancer Res 48: 4361-7 (1988).
7. Fukushi Y, et al. Biochemistry 25: 2859-66 (1986).
8. Fukushi Y, et al. JBC 259: 10511-10517 (1984)
9. Ito A, et al. JBC2 76(20): 16695-703 (2001).
10. Saito S, et al. JBC 269: 5644-52 (1994).

Tumor-associated glycosyl epitopes associated with haptoglobin alpha and beta chains

| | | | Sialyl Le^{x}-Le^{x} | | Sialyl Le^{x} | | Sialyl Le^{a} | | Sialyl Tn | |
|---|---|---|---|---|---|---|---|---|---|---|
| Sugar | CBB | | FH6 | | SNH3 | | NS19-9 | | 47 TKH | |
| Antibody | beta | alpha | beta | alpha | beta | alpha | beta | alpha | beta | alpha |
| Haptoglobin | 37 kDa | 17 kDa | 37 kDa | 17 kDa | 37 kDa | 17 kDa | 37 kDa | 17 kDa | 37 kDa | 17 kDa |
| Normal | | | | | | | | | | |
| health | | | | | | | | | | |
| M | − | − | − | − | − | + | − | − | − | − |
| F | − | − | − | − | − | + | − | − | +/− | − |
| Cancer | | | | | | | | | | |
| L2 | +++ | ++ | + | ++ | − | ++++++ | ++ | + | ++ | ++ |
| L5 | − | +/− | +/− | +/− | − | ++++ | − | − | + | + |
| L8 | +++ | ++ | + | ++ | − | ++++++ | +++ | +++ | +++ | +++ |
| B2 | − | − | − | − | − | − | − | − | + | − |
| B3 | ++ | +/− | + | +/− | − | ++++ | ++ | + | ++ | ++ |
| G2A | − | − | − | − | − | ++ | +/− | − | + | − |
| C1A | − | +/− | − | +/− | − | +++ | + | + | + | +/− |
| M3 | ++ | + | + | +/− | − | +++ | ++ | +++ | ++ | ++ |
| M8 | ++ | ++ | + | +/− | − | +++ | ++ | +++ | ++ | ++ |

L2 Small cell lung carcinoma middle stage
L5 Adenocarcinoma early stage
L8 Squamous cell carcinoma middle stage
B2 Breast cancer early stage
G2A Gastric cancer early stage
C1A Sigmoid colon cancer middle stage
M3 Prostate cancer malignant
M8 Prostate cancer malignant

We claim:

1. A method for determining the presence of two or more tumor-associated antigens in a biological sample, comprising:
providing a Surface Plasmon Resonance (SPR) system having a plurality of channels, each channel having an antibody specific for a tumor-associated antigen operably affixed to a surface thereof;
applying a biological sample from a patient to said SPR system; and
measuring an SPR signal shift from each of said channels, to thereby determine the presence of said two or more tumor-associated antigens in the biological sample.

2. The method of claim 1, wherein at least one of said two or more tumor-associated antigens is a carbohydrate antigen selected from the group consisting of Le^{x}, dimeric Le^{x}, sialyl Le^{x}, sialyl Le^{a}, sialyl Tn, Tn, disialyl Lc₄, sialyl dimeric Le^{x}, and GalNAc disialo Lc₄.

3. The method of claim 2, wherein each of said multiple channels further has operably affixed to the surface thereof, an antibody specific for a polypeptide to which the carbohydrate antigen is associated.

4. The method of claim 3, wherein the carbohydrate antigen is Sialyl Le^{x} and the antibody specific for a polypeptide to which said Sialyl Le^{x} is associated is haptoglobin alpha 2 chain.

5. The method of claim 1, wherein each said antibody is a Fab fragment.

6. The method of claim 1, wherein said biological sample is a serum sample.

7. The method of claim 1, wherein said patient is suspected of having lung cancer, breast cancer, gastric cancer, or prostate cancer.

8. The method of claim 1, wherein each said antibody is operably affixed to the surface through a self-assembling monolayer (SAM).

9. The method of claim 1, wherein each said channel has a sensor area and a self-referencing area,
said sensor area having operably affixed thereto said antibody specific for a tumor-associated antigen, and
said self-referencing area having operably fixed thereto a control antibody to control for non-specific binding events and environmental changes.

10. The method of claim 9, wherein said SPR system comprises a digital window system controlling illumination of the channels, and allowing for sequential measurement of the SPR signal shift of each of said channels.

11. A self-referencing Surface Plasmon Resonance (SPR) system for determining the presence of two or more tumor-associated antigens in a biological sample, comprising:
(a) an SPR system having a sensor surface, said sensor surface having multiple channels, each channel having operably affixed thereto an antibody specific for a tumor associated antigen;
(b) a mechanism for applying a flow of sample to the sensor surface, said flow of sample resulting in an antigen-antibody interaction on the sensor surface when antigen is present, and causing a shift in an SPR signal.

12. The SPR system of claim 11, wherein at least one of said two or more tumor-associated antigens is a carbohydrate antigen selected from the group consisting of Le^{x}, dimeric Le^{x}, sialyl Le^{x}, sialyl Le^{a}, sialyl Tn, Tn, disialyl Lc₄, sialyl dimeric Le^{x}, and GalNAc disialo Lc₄.

13. The SPR system of claim 12, wherein each of said multiple channels further has operably affixed to the surface thereof, an antibody specific for a polypeptide to which the carbohydrate antigen is associated.

14. The SPR system of claim 13, wherein the carbohydrate antigen is Sialyl Le^{x} and the antibody specific for a polypeptide to which said Sialyl Le^{x} is associated is haptoglobin alpha 2 chain.

15. The SPR system of claim 11, wherein each said antibody is a Fab fragment.

16. The SPR system of claim 11, wherein the presence of each of said tumor-associated antigens is determined on its own parallel channel, each parallel channel having a sensing area and a self-referencing area, each sensing area having said antibody specific for a tumor-associated antigen operably affixed thereto, and each self-referencing area having operably affixed thereto a control antibody to control for non-specific and environmental changes.

17. The SPR system of claim 11, wherein each said antibody specific for a tumor-associated antigen is affixed to the sensor surface by a SAM comprising 16-mercaptohexadecanoic acid and/or 11-mercaptoundecanol.

18. The SPR system of claim 11, wherein said mechanism for applying flow of sample comprises a sample cassette, said sample cassette having a place for insertion of a sensor chip.

19. The SPR system of claim 11, wherein said SPR system uses a digital window system with electrochromic organic polymers that change color when voltage is applied, the digital window system keeping each channel in an on or off state by controlling illumination of said channels.

20. The method of claim 11, wherein the intensity of reflected light from each channel is monitored by CCD camera or photodiode array.

21. The SPR system of claim 11, wherein said SPR system comprises a monochromatic light source selected from He-Ne laser or laser emitting diode (LED).

22. The SPR system of claim 11, further comprising a polarizer, a lens and a dove-type or semi-cylindrical glass prism.
